(19) United States
(12) Patent Application Publication
(10) Pub. No.: US 2016/0106821 A1
(43) Pub. Date: Apr. 21, 2016

(54) FUNCTIONAL GHRELIN RECEPTOR ANTAGONISM DURING PREGNANCY TO PREVENT STRESS-ASSOCIATED MENTAL ILLNESS IN OFFSPRING AND MOTHERS

(71) Applicant: Massachusetts Institute of Technology, Cambridge, MA (US)

(72) Inventor: Ki Ann Goosens, Cambridge, MA (US)

(73) Assignee: Massachusetts Institute of Technology, Cambridge, MA (US)

(21) Appl. No.: 14/887,573

(22) Filed: Oct. 20, 2015

Related U.S. Application Data
(60) Provisional application No. 62/065,987, filed on Oct. 20, 2014.

(51) Int. Cl. A61K 39/00; C12Q 1/68; G01N 33/68; A61K 38/45

(52) U.S. Cl. CPC A61K 39/0005; A61K 38/45; C12Q 1/6883; G01N 33/6896; C12Y 203/01; C12Q 2600/158; G01N 2800/28

(57) ABSTRACT

The invention relates to methods of protecting against elevated ghrelin levels in a fetus by antagonizing ghrelin or ghrelin receptor. The invention also encompasses methods of treating postpartum depression by administering ghrelin antagonists.

### RELATED APPLICATIONS

This application claims priority under 35 U.S.C. §119(e) to U.S. Provisional Application Ser. No 62/065,987, filed on Oct. 20, 2014, which is herein incorporated by reference in its entirety.

### GOVERNMENT INTEREST

This invention was made with government support under Grant No. MH084966 awarded by the National Institutes of Health and under Contact No. W911NF-10-1-0059 awarded by the Army Research Office. The government has certain rights in the invention.

### FIELD OF THE INVENTION

The invention relates to antagonism of ghrelin or ghrelin receptor to protect against, treat or prevent fetal disorders and postpartum depression.

BACKGROUND INFORMATION

Ghrelin is a peptide hormone produced primarily by gastrointestinal cells. Receptors for ghrelin are highly expressed in regions of the hypothalamus that control feeding. Accordingly, ghrelin has been extensively studied for its ability to induce feeding behavior. However, ghrelin receptors are also expressed in other brain regions not traditionally associated with feeding behavior, such as the hippocampus. Ghrelin signaling is linked to obesity, diabetes and cardiovascular function.

### SUMMARY OF INVENTION

Aspects of the invention relate to a method for treating stress induced susceptibility to mental disorders in a fetus, by determining a level of ghrelin expression in a pregnant subject and if the pregnant subject has ghrelin levels that are higher than normal ghrelin levels in a normal pregnant subject at a similar stage of pregnancy, administering to the pregnant subject an effective amount of a ghrelin antagonist to reduce functional ghrelin exposure in a fetus and to treat stress induced susceptibility to mental disorders in the fetus.

In some embodiments the pregnant subject has not previously been identified as a subject that is exposed to chronic stress. In other embodiments the pregnant subject has not previously been identified as a subject that was previously exposed to chronic stress.

The method may also involve further continuing to treat the pregnant subject after the fetus is born. In other embodiments the method may further involve once the fetus is born, administering to the baby an effective amount of a ghrelin antagonist.

In some embodiments, the ghrelin antagonist targets the ghrelin receptor. In certain embodiments, the ghrelin antagonist is a GHSr1a antagonist or a GHSr1a inverse antagonist. In other embodiments, the ghrelin antagonist targets ghrelin. In certain embodiments, the ghrelin antagonist is an anti-ghrelin vaccine. In other embodiments, the ghrelin antagonist targets ghrelin O-acyltransferase (GOAT). In certain embodiments, the ghrelin antagonist is an anti-GOAT vaccine. In other embodiments, the ghrelin antagonist is a compound that reduces or inhibits the synthesis or release of ghrelin by the stomach and other tissues. In other embodiments, the ghrelin antagonist is a compound that reduces or prevents ghrelin from crossing the blood-brain barrier. In other embodiments the treatment involves reducing levels in circulation using agents that inactivate (deacylate) ghrelin or by agents that increase plasma esterases responsible for endogenous ghrelin deacylation, such as APT1 and other putative esterases. Thus, esterases such as APT1 are agents targeting ghrelin or the ghrelin receptor according to the invention. Agents that increase the breakdown of ghrelin such as vaccines are also ghrelin antagonists of the invention.

In some embodiments the ghrelin antagonist is a compound that reduces or inhibits the synthesis or release of ghrelin by the stomach and other tissues. In other embodiments the ghrelin antagonist is a compound that reduces or prevents ghrelin from crossing the blood-brain barrier.

In another aspect the invention is method for identifying a fetus at risk of developing a mental disorder, by determining a level of ghrelin expression in a pregnant subject and comparing the ghrelin levels with normal ghrelin levels found in a normal pregnant subject at a similar stage of pregnancy, wherein if the pregnant subject has ghrelin levels that are higher than normal ghrelin levels, the pregnant subject has a fetus that is at risk of developing a mental disorder.

In an embodiment normal ghrelin levels correspond to ghrelin levels in the pregnant subject prior to pregnancy. In another embodiment the level of ghrelin expression is assayed on a blood sample from the pregnant subject.

The invention in other aspects is a method for treating postpartum depression in a subject, by administering to a subject having or susceptible to postpartum depression an effective amount of a ghrelin antagonist to treat the postpartum depression in the subject.

In some embodiments the subject has elevated ghrelin levels, relative to a normal subject. In other embodiments the subject has not previously been identified as a subject that is exposed to chronic stress. In yet other embodiments the subject has not previously been identified as having elevated ghrelin levels.

These and other aspects of the invention, as well as various embodiments thereof, will become more apparent in reference to the drawings and detailed description of the invention.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a graph depicting depressive behaviors in post-partum mothers.

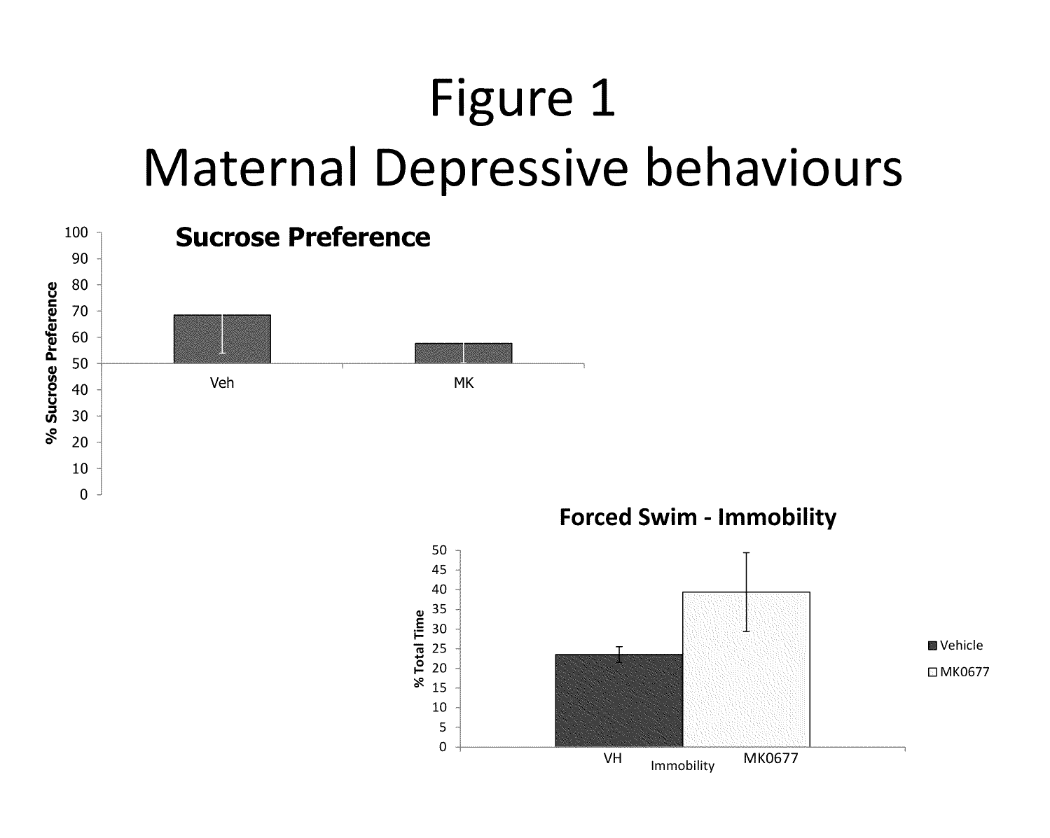

FIG. 2 is a diagram depicting the protocol for analysis of babies following birth.

FIG. 3 depicts the design of the study with protocols involving vehicle control, low MK0677 or high MK0677 treatment.

FIG. 4 is a graph depicting the effects of perinatal exposure to MK0677 on female (dark bar) versus male (lighter gray bar) offspring. V refers to a vehicle control. H refers to high MK0677 exposure levels.

FIG. 5 is a graph depicting the effects of exposure to MK0677 in the womb on offspring.

FIG. 6 is a bar graph depicting the influence of gestational exposure to MK0677 on exploratory behaviors of female (light gray bar) versus male (dark gray bar) offspring as measured in Open Field inner arena. V refers to a vehicle control. M refers to MK0677.

FIGS. 7A and 7B are bar graphs depicting the influence of gestational exposure to MK0677 on exploratory behaviors of female (FIG. 7A) versus male (FIG. 7B) offspring as measured in an Elevated Plus Maze (EPM) Open Arm. The Distal Open Arm is shown in the top dark gray bar and the Medial Open Arm is shown in the lower light gray bar. V refers to a vehicle control. M refers to MK0677.

### DETAILED DESCRIPTION

The invention is based, at least in part, on the surprising discovery that elevated levels of ghrelin in a pregnant subject have long lasting implications on susceptibility to mental disorders in the fetus. In particular, the effects in the fetus remain after birth through adulthood. It was discovered according to the invention that a fetus exposed to elevated maternal ghrelin levels have elevated ghrelin levels and associated altered behaviors as adults as a result of the elevated maternal ghrelin levels. In particular subjects that have been exposed to elevated maternal ghrelin levels demonstrate more risk-taking behaviors, including, for instance hyperexploratory behaviors and more anhedonic behaviors. Risk taking behaviors are associated with a higher propensity for addiction or thrill seeking. Anhedonic subjects tend to undervalue rewards and overestimate aversive stimuli.

Accordingly, therapeutic and prophylactic approaches based on antagonism of ghrelin or ghrelin receptor can be used in a pregnant mother to prevent or reduce the incidence of stress-induced susceptibility to mental disorders long after the fetus is born. Further described herein are methods for treating postpartum depression in pregnant women by antagonizing ghrelin in those women.

Methods associated with the invention comprise administration of a ghrelin antagonist signaling to a subject. A ghrelin antagonist, as used herein is a functional antagonist that is an agent that inhibits the level or activity of a component of the ghrelin signaling pathway. Ghrelin antagonists are referred to herein as agents or agents that inhibit the level of activity of ghrelin or ghrelin receptor or as functional antagonists of ghrelin signaling. For example, an agent can target ghrelin itself, or the ghrelin receptor or can target one or more other factors which influence the level or activity of ghrelin, such as ghrelin O-acyltransferase (GOAT) or a downstream effector such as growth hormone. For example, the agent can be a vaccine, such as a vaccine against ghrelin, ghrelin receptor or GOAT. The agent may also be a receptor inverse agonist or a compound that degrades ghrelin before it even gets to the receptor. Anything that interferes with ghrelin receptor activation, whether it directly acts on the receptor or not is a ghrelin antagonist.

In certain embodiments, the ghrelin antagonist is an antagonist of the ghrelin receptor, which also include: a ghrelin antibody or antigen-binding fragment thereof, a ghrelin derivative, a ghrelin inhibitor, a ghrelin receptor peptide or fragment, a ghrelin receptor inhibitor, a ghrelin receptor antibody or antigen-binding fragment thereof, a ghrelin analog, a ghrelin receptor peptide or fragment and a non-peptide ghrelin receptor antagonist. Examples of antagonist of the ghrelin receptor include a GHSr1a antagonist or a GHSr1a inverse antagonist. Other examples of antagonist of the ghrelin receptor include but are not limited to

Gly-Ser-Ser(Octanoyl)-Phe-A; where A is —OH, NH₂ (SEQ ID NO: 1), Leu-Ser-Pro-Glu-X (SEQ ID NO: 2), or -Ala-Lys-Leu-Gln-Pro-Arg- —B where B is —OH or NH₂ (SEQ ID NO: 3)

Gly-Ser-Ser(Octanoyl)-Phe-Leu-Ser-Pro-Glu (SEQ ID NO: 4)

[D-lys-3]-GHRP-6 (His-D-Trp-D-Lys-Trp-D-Phe-Lys-NH₂)

L-756867 (i.e. D-ArgPro-Lys-Pro-D-Phe-Gln-D-Trp-Phe-D-Trp-Leu-Leu-NH₂

substance P derivative; (D-Lys3)-GHRP-6 (i.e. His-D-Trp-D-Lys-Trp-D-Phe-Lys-NH₂

non-peptidyl antagonist denoted L-692400

cyclo(-His-D-Trp-Ala-Trp-D-Phe-)

(2E)-4-(1-aminocyclobutyl)but-2-enoic acid N-((1R)-1-diphenethylcarbamoyl-2-(2-naphthyl)ethyl)-N-methylamide

(E)-5-amino-5-methylhex-2-enoic [(R)-2-(1-(benzofuran-7-yl)-7-chloro-8-methoxy-1,2,4,5-tetrahydrobenzo[d]-azepin-3-yl)-1-(benzyloxymethyl)-2-oxoethyl]amide

2-amino-N-[(1R)-1-{N-[(1R)-1-(N′,N′-dimethylhydrazinocarbonyl)-3-phenylpropyl]-N-methylcarbamoyl}-2-(1H-indol-3-yl)ethyl]-2-methylpropionamide, or

2-[(1R)-1-((2E)-5-amino-5-methylhex-2-enoylamino)-2-(2-naphthyl)ethyl-]-5-phenyloxazole-4-carboxylic acid methyl ester.

The GHSR is encoded by a single gene found at chromosomal location 3q26.2. Alternative mRNA processing generates 2 types of GHSR proteins: GHSR1a and GHSR1b. GHSR1a is a G-protein-linked receptor consisting of 366 amino acids with 7 transmembrane regions. Stimulation of GHSR1a by GHSs or ghrelin triggers the phospholipase C signaling pathway, leading to increased inositol phosphate turnover and protein kinase C activation, resulting in the release of calcium from intracellular stores. GHSR activation also inhibits K channels, allowing the entry of calcium through voltage-gated l- and T-type channels. In contrast, GHSR1b consists of 289 amino acids with only 5 transmembrane domains. The antagonist of ghrelin receptor may antagonize either GHSR1a or GHSR1b or both.

The ghrelin antagonist can also be a compound that inhibits the synthesis or release of ghrelin in the stomach. The agent may also be a compound that reduces or prevents ghrelin from crossing the blood-brain barrier. The agent may also be an esterase such as APT1.

The agent can also be an inhibitory nucleic acid, such as an siRNA or an antisense molecule that inhibits expression of a ghrelin signaling component. The nucleic acid sequence of ghrelin is known in the art. See for instance, Gene ID: 51738 in NCBI database. The inhibitory nucleic acids may be designed using routine methods in the art.

A ghrelin inhibitory nucleic acid typically causes specific gene knockdown, while avoiding off-target effects. Various strategies for gene knockdown known in the art can be used to inhibit gene expression. For example, gene knockdown strategies may be used that make use of RNA interference (RNAi) and/or microRNA (miRNA) pathways including small interfering RNA (siRNA), short hairpin RNA (shRNA), double-stranded RNA (dsRNA), miRNAs, and other small interfering nucleic acid-based molecules known in the art. In one embodiment, vector-based RNAi modalities (e.g., shRNA or shRNA-mir expression constructs) are used to reduce expression of a gene (e.g., a target nucleic acid such as a ghrelin nucleic acid) in a cell. In some embodiments, therapeutic compositions of the invention comprise an isolated plasmid vector (e.g., any isolated plasmid vector known in the art or disclosed herein) that expresses a small interfering nucleic acid such as an shRNA. The isolated plasmid may comprise a specific promoter operably linked to a gene encoding the small interfering nucleic acid. In some cases, the isolated plasmid vector is packaged in a virus capable of infecting the individual. Exemplary viruses include adenovirus, retrovirus, lentivirus, adeno-associated virus, and others that are known in the art and disclosed herein.

A broad range of RNAi-based modalities could be employed to inhibit expression of a gene in a cell, such as siRNA-based oligonucleotides and/or altered siRNA-based oligonucleotides. Altered siRNA based oligonucleotides are those modified to alter potency, target affinity, safety profile and/or stability, for example, to render them resistant or partially resistant to intracellular degradation. Modifications, such as phosphorothioates, for example, can be made to oligonucleotides to increase resistance to nuclease degradation, binding affinity and/or uptake. In addition, hydrophobization and bioconjugation enhances siRNA delivery and targeting (De Paula et al., RNA. 13(4):431-56, 2007) and siRNAs with ribo-difluorotoluyl nucleotides maintain gene silencing activity (Xia et al., ASC Chem. Biol. 1(3):176-83, (2006)). siRNAs with amide-linked oligoribonucleosides have been generated that are more resistant to S1 nuclease degradation than unmodified siRNAs (Iwase R et al. 2006 Nucleic Acids Symp Ser 50: 175-176). In addition, modification of siRNAs at the 2′-sugar position and phosphodiester linkage confers improved serum stability without loss of efficacy (Choung et al., Biochem. Biophys. Res. Commun. 342(3):919-26, 2006). Other molecules that can be used to inhibit expression of a gene (e.g., a CSC-associated gene) include sense and antisense nucleic acids (single or double stranded), ribozymes, peptides, DNAzymes, peptide nucleic acids (PNAs), triple helix forming oligonucleotides, antibodies, and aptamers and modified form(s) thereof directed to sequences in gene(s), RNA transcripts, or proteins. Antisense and ribozyme suppression strategies have led to the reversal of a tumor phenotype by reducing expression of a gene product or by cleaving a mutant transcript at the site of the mutation (Carter and Lemoine Br. J. Cancer. 67(5):869-76, 1993; Lange et al., Leukemia. 6(11):1786-94, 1993; Valera et al., J. Biol. Chem. 269(46):28543-6, 1994; Dosaka-Akita et al., Am. J. Clin. Pathol. 102(5):660-4, 1994; Feng et al., Cancer Res. 55(10):2024-8, 1995; Quattrone et al., Cancer Res. 55(1):90-5, 1995; Lewin et al., Nat Med. 4(8):967-71, 1998). Ribozymes have also been proposed as a means of both inhibiting gene expression of a mutant gene and of correcting the mutant by targeted trans-splicing (Sullenger and Cech Nature 371(6498):619-22, 1994; Jones et al., Nat. Med. 2(6):643-8, 1996). Ribozyme activity may be augmented by the use of, for example, non-specific nucleic acid binding proteins or facilitator oligonucleotides (Herschlag et al., Embo J. 13(12):2913-24, 1994; Jankowsky and Schwenzer Nucleic Acids Res. 24(3):423-9,1996). Multitarget ribozymes (connected or shotgun) have been suggested as a means of improving efficiency of ribozymes for gene suppression (Ohkawa et al., Nucleic Acids Symp Ser. (29):121-2, 1993).

Other inhibitor molecules that can be used include sense and antisense nucleic acids (single or double stranded). Antisense nucleic acids include modified or unmodified RNA, DNA, or mixed polymer nucleic acids, and primarily function by specifically binding to matching sequences resulting in modulation of peptide synthesis (Wu-Pong, November 1994, BioPharm, 20-33). Antisense nucleic acid binds to target RNA by Watson Crick base-pairing and blocks gene expression by preventing ribosomal translation of the bound sequences either by steric blocking or by activating RNase H enzyme. Antisense molecules may also alter protein synthesis by interfering with RNA processing or transport from the nucleus into the cytoplasm (Mukhopadhyay & Roth, 1996, Crit. Rev. in Oncogenesis 7, 151-190).

As used herein, the term “antisense nucleic acid” describes a nucleic acid that is an oligoribonucleotide, oligodeoxyribonucleotide, modified oligoribonucleotide, or modified oligodeoxyribonucleotide which hybridizes under physiological conditions to DNA comprising a particular gene or to an mRNA transcript of that gene and, thereby, inhibits the transcription of that gene and/or the translation of that mRNA. The antisense molecules are designed so as to interfere with transcription or translation of a target gene upon hybridization with the target gene or transcript. Those skilled in the art will recognize that the exact length of the antisense oligonucleotide and its degree of complementarity with its target will depend upon the specific target selected, including the sequence of the target and the particular bases which comprise that sequence.

In some embodiments the inhibitory nucleic acid of the invention is 100% identical to the nucleic acid target. In other embodiments it is at least 99%, 95%, 90%, 85%, 80%, 75%, 70%, or 50% identical to the nucleic acid target. The term “percent identical” refers to sequence identity between two nucleotide sequences. Percent identity can be determined by comparing a position in each sequence which may be aligned for purposes of comparison. Expression as a percentage of identity refers to a function of the number of identical amino acids or nucleic acids at positions shared by the compared sequences. Various alignment algorithms and/or programs may be used, including FASTA, BLAST, or ENTREZ-FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

An inhibitory nucleic acid useful in the invention will generally be designed to have partial or complete complementarity with one or more target genes (i.e., complementarity with one or more transcripts of ghrelin gene). The target gene may be a gene derived from the cell, an endogenous gene, a transgene, or a gene of a pathogen which is present in the cell after infection thereof. Depending on the particular target gene, the nature of the inhibitory nucleic acid and the level of expression of inhibitory nucleic acid (e.g. depending on copy number, promoter strength) the procedure may provide partial or complete loss of function for the target gene. Quantitation of gene expression in a cell may show similar amounts of inhibition at the level of accumulation of target mRNA or translation of target protein.

“Inhibition of gene expression” refers to the absence or observable decrease in the level of protein and/or mRNA product from a target gene. “Specificity” refers to the ability to inhibit the target gene without manifest effects on other genes of the cell. The consequences of inhibition can be confirmed by examination of the outward properties of the cell or organism or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS). For RNA-mediated inhibition in a cell line or whole organism, gene expression is conveniently assayed by use of a reporter or drug resistance gene whose protein product is easily assayed. Such reporter genes include acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentamycin, hygromycin, kanamycin, lincomycin, methotrexate, phosphinothricin, puromycin, and tetracyclin.

Depending on the assay, quantitation of the amount of gene expression allows one to determine a degree of inhibition which is greater than 10%, 33%, 50%, 90%, 95% or 99% as compared to a cell not treated according to the present invention. As an example, the efficiency of inhibition may be determined by assessing the amount of gene product in the cell: mRNA may be detected with a hybridization probe having a nucleotide sequence outside the region used for the inhibitory nucleic acid, or translated polypeptide may be detected with an antibody raised against the polypeptide sequence of that region.

The ghrelin antagonist can be an agent that has been developed to antagonize ghrelin in other contexts, such as to combat obesity or diabetes. Several non-limiting examples of commercially available agents that antagonize ghrelin signaling include: small molecule ghrelin receptor antagonists from Elixir Pharmaceuticals, ghrelin antagonist AEZS-123 from AEterna Zentaris Inc., anti-ghrelin vaccine from Cytos Biotechnology, ghrelin receptor antagonists from Merck, ghrelin antagonists from Tranzyme Pharma, small molecule ghrelin receptor antagonists from Bayer, ghrelin receptor antagonist DLys3 GHRP-6 from Phoenix Pharmaceuticals, humanized anti-ghrelin antibodies from Eli Lilly and Company and ghrelin binding nucleic acids that antagonize ghrelin activity from Noxxon Pharma AG, each of which is an agent that inhibits the level of activity of ghrelin or ghrelin receptor, as used herein.

Non-limiting examples of agents for inhibiting ghrelin signaling are found in, and expressly incorporated by reference for their teachings related to agents for inhibiting ghrelin signaling from, US Patent publication numbers: US20110318807, US20110257086, US20110245161, US20110245160, US20110021420, US20100286152, US20100254994, US20100196396, US20100196330, US20100086955, US20100021487, US20090275648, US20090253673, US20090149512, US20070275877, US20070237775, US20070025991, US20050201938, US20050191317, US20050070712 and US20020187938, and from U.S. Pat. Nos. 8,013,015, 7,901,679, 7,666,833 and 7,479,271.

The ghrelin antagonist is administered to a subject. Typically the subject is a pregnant subject. A pregnant subject is used to refer to a subject that is carrying at least one fetus. In some instances the term subject is used to refer to a subject that plans to or is at risk of getting pregnant and has high ghrelin levels. In that instance the pregnant subject is said to be a subject who may be pregnant or who is capable of being pregnant. In other instances the term pregnant subject may refer to a subject that has been pregnant but has recently given birth to a baby, within the previous 6 months. The subject may also be a subject having or susceptible to postpartum depression. A subject having postpartum depression is a subject that has given birth to a baby within the prior two years and who has high levels of ghrelin. A subject susceptible to postpartum depression is a subject who may be pregnant or have given birth and who has at least one risk factor associated with postpartum depression.

The ghrelin antagonist can be administered to the pregnant subject before, during and/or after the fetus is conceived or born. For example, the agent can be administered to a subject in anticipation of pregnancy, when ghrelin levels are found to be high. As such, the agent can protect against the consequences of fetal exposure to ghrelin once conception occurs. The agent can also be administered to a subject during the pregnancy to protect against the consequences of exposure to ghrelin throughout the remainder of the pregnancy. The agent can also be administered after the pregnancy to protect the baby through reduction of ghrelin in breast milk and passage of the functional ghrelin antagonist to the baby through the milk.

The level of ghrelin in a subject is typically compared to a control level. It should be appreciated that the appropriate control will vary depending on the circumstances. In some embodiments, the control level can be the level of ghrelin in the same subject prior to exposure to chronic stress. In other embodiments, the control level can be the level of ghrelin in a pregnant subject who has normal levels of ghrelin. Levels of ghrelin may be measured at multiple time points and may be measured before, during and after pregnancy.

In its broadest sense, the terms “treatment” or “to treat” refer to both therapeutic and prophylactic treatments. If the subject or fetus in need of treatment has already been exposed to high ghrelin levels, then “treating the condition” refers to ameliorating, reducing or eliminating the high levels of ghrelin within the fetus or reducing the severity of a postpartum depression or preventing any further progression of this disease. If the subject in need of treatment is one who is at risk of having a condition, then treating the subject refers to reducing the risk of the subject having the condition or preventing the subject from developing the condition.

A subject shall mean a human or vertebrate animal or mammal including but not limited to a dog, cat, horse, cow, pig, sheep, goat, turkey, chicken, and primate, e.g., monkey.

Therapeutic compounds associated with the invention may be directly administered to the subject or may be administered in conjunction with a delivery device or vehicle. Delivery vehicles or delivery devices for delivering therapeutic compounds to surfaces have been described. The therapeutic compounds of the invention may be administered alone (e.g., in saline or buffer) or using any delivery vehicles known in the art.

The term effective amount of a therapeutic compound of the invention refers to the amount necessary or sufficient to realize a desired biologic effect. For example, an effective amount of a therapeutic compound associated with the invention may be that amount sufficient to ameliorate the stress induced susceptibility to mental disorders in the fetus. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject. The effective amount for any particular application can vary depending on such factors as the disease or condition being treated, the particular therapeutic compounds being administered the size of the subject, or the severity of the disease or condition. One of ordinary skill in the art can empirically determine the effective amount of a particular therapeutic compound associated with the invention without necessitating undue experimentation.

Subject doses of the compounds described herein for delivery typically range from about 0.1 μg to 10 mg per administration, which depending on the application could be given daily, weekly, or monthly and any other amount of time there between. The doses for these purposes may range from about 10 μg to 5 mg per administration, and most typically from about 100 μg to 1 mg, with 2-4 administrations being spaced days or weeks apart. In some embodiments, however, parenteral doses for these purposes may be used in a range of 5 to 10,000 times higher than the typical doses described above.

It was discovered that the doses necessary to reduce dangerous ghrelin levels in a fetus are significantly lower than the dose needed to alter food consumption. In some instances, a therapeutically effective amount of an agent of the invention is 100-1,000 times lower than a typical dose used for altering food consumption. In other embodiments, a therapeutically effective amount of an agent of the invention is 10,000 times lower than a typical dose used for altering food consumption. In some embodiments a compound of the invention is administered at a dosage of between about 1 and 10 mg/kg of body weight of the mammal. In other embodiments a compound of the invention is administered at a dosage of between about 0.001 and 1 mg/kg of body weight of the mammal. In yet other embodiments a compound of the invention is administered at a dosage of between about 10-100 ng/kg, 100-500 ng/kg, 500 ng/kg-1 mg/kg, or 1-5 mg/kg of body weight of the mammal, or any individual dosage therein.

The formulations of the invention are administered in pharmaceutically acceptable solutions, which may routinely contain pharmaceutically acceptable concentrations of salt, buffering agents, preservatives, compatible carriers, and optionally other therapeutic ingredients.

For use in therapy, an effective amount of the therapeutic compound associated with the invention can be administered to a subject by any mode that delivers the therapeutic agent or compound to the desired surface, e.g., mucosal, systemic. Administering the pharmaceutical composition of the present invention may be accomplished by any means known to the skilled artisan. Preferred routes of administration include but are not limited to oral, parenteral, intramuscular, intranasal, sublingual, intratracheal, inhalation, ocular, vaginal, rectal and intracerebroventricular.

For oral administration, the therapeutic compounds of the invention can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, i.e., EDTA for neutralizing internal acid conditions or may be administered without any carriers.

Also specifically contemplated are oral dosage forms of the above component or components. The component or components may be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline (Abuchowski and Davis, 1981, “Soluble Polymer-Enzyme Adducts” In: Enzymes as Drugs, Hocenberg and Roberts, eds., Wiley-Interscience, New York, N.Y., pp. 367-383; Newmark, et al., 1982, J. Appl. Biochem. 4:185-189). Other polymers that could be used are poly-1,3-dioxolane and poly-1,3,6-tioxocane. Preferred for pharmaceutical usage, as indicated above, are polyethylene glycol moieties.

The location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the therapeutic agent or by release of the biologically active material beyond the stomach environment, such as in the intestine.

To ensure full gastric resistance a coating impermeable to at least pH 5.0 is preferred. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic i.e., powder; for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multi-particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the therapeutic agent may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, a-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential non-ionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the therapeutic agent either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

Also contemplated herein is pulmonary delivery of the therapeutic compounds of the invention. The therapeutic agent is delivered to the lungs of a mammal while inhaling and traverses across the lung epithelial lining to the blood stream. Other reports of inhaled molecules include Adjei et al., 1990, Pharmaceutical Research, 7:565-569; Adjei et al., 1990, International Journal of Pharmaceutics, 63:135-144 (leuprolide acetate); Braquet et al., 1989, Journal of Cardiovascular Pharmacology, 13(suppl. 5):143-146 (endothelin-1); Hubbard et al., 1989, Annals of Internal Medicine, Vol. III, pp. 206-212 (a1-antitrypsin); Smith et al., 1989, J. Clin. Invest. 84:1145-1146 (a-l-proteinase); Oswein et al., 1990, “Aerosolization of Proteins”, Proceedings of Symposium on Respiratory Drug Delivery II, Keystone, Colo., March, (recombinant human growth hormone); Debs et al., 1988, J. Immunol. 140:3482-3488 (interferon-g and tumor necrosis factor alpha) and Platz et al., U.S. Pat. No. 5,284,656 (granulocyte colony stimulating factor). A method and composition for pulmonary delivery of drugs for systemic effect is described in U.S. Pat. No. 5,451,569, issued Sep. 19, 1995 to Wong et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass.

All such devices require the use of formulations suitable for the dispensing of therapeutic agent. Typically, each formulation is specific to the type of device employed and may involve the use of an appropriate propellant material, in addition to the usual diluents, and/or carriers useful in therapy. Also, the use of liposomes, microcapsules or microspheres, inclusion complexes, or other types of carriers is contemplated. Chemically modified therapeutic agent may also be prepared in different formulations depending on the type of chemical modification or the type of device employed.

Formulations suitable for use with a nebulizer, either jet or ultrasonic, will typically comprise therapeutic agent dissolved in water at a concentration of about 0.1 to 25 mg of biologically active compound per mL of solution. The formulation may also include a buffer and a simple sugar (e.g., for stabilization and regulation of osmotic pressure). The nebulizer formulation may also contain a surfactant, to reduce or prevent surface induced aggregation of the compound caused by atomization of the solution in forming the aerosol.

Formulations for use with a metered-dose inhaler device will generally comprise a finely divided powder containing the therapeutic agent suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing therapeutic agent and may also include a bulking agent, such as lactose, sorbitol, sucrose, or mannitol in amounts which facilitate dispersal of the powder from the device, e.g., 50 to 90% by weight of the formulation. The therapeutic agent should most advantageously be prepared in particulate form with an average particle size of less than 10 mm (or microns), most preferably 0.5 to 5 mm, for most effective delivery to the distal lung.

Intra-nasal delivery of a pharmaceutical composition of the present invention is also contemplated. Intra-nasal delivery allows the passage of a pharmaceutical composition of the present invention to the blood stream directly after administering the therapeutic product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

Ghrelin antagonists associated with the invention can be formulated as vaccines, such as an anti-ghrelin vaccine or an anti-GOAT vaccine. Preferably, prophylactic vaccination is used in pregnant subjects that are not diagnosed with a condition such as a stress-sensitive condition, and more preferably the subjects are considered at risk of developing a condition such as a stress-sensitive condition. For example, the subject may be administered a vaccine, such as an anti-ghrelin vaccine or an anti-GOAT vaccine before pregnancy. Vaccines can be administered through any means familiar to one or ordinary skill in the art. For example, vaccines can be administered by immersion or orally.

Vaccines in some instances activate the humoral immune system (i.e., the antibody dependent immune response). Other vaccines activate the cell-mediated immune system including cytotoxic T lymphocytes.

The agents, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990, which is incorporated herein by reference.

The therapeutic compounds of the invention and optionally other therapeutics may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The pharmaceutical compositions of the invention contain an effective amount of a therapeutic compound of the invention optionally included in a pharmaceutically-acceptable carrier. The term pharmaceutically-acceptable carrier means one or more compatible solid or liquid filler, diluents or encapsulating substances which are suitable for administration to a human or other vertebrate animal. The term carrier denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

The present invention is further illustrated by the following Examples, which in no way should be construed as further limiting. The entire contents of all of the references (including literature references, issued patents, published patent applications, and co-pending patent applications) cited throughout this application are hereby expressly incorporated herein by reference.

### EXAMPLES

It was discovered herein that elevated ghrelin signaling during pregnancy leads to changes in emotional processing in offspring. As a result ghrelin receptor antagonism in pregnant females may block stress induced susceptibility to mental disorders.

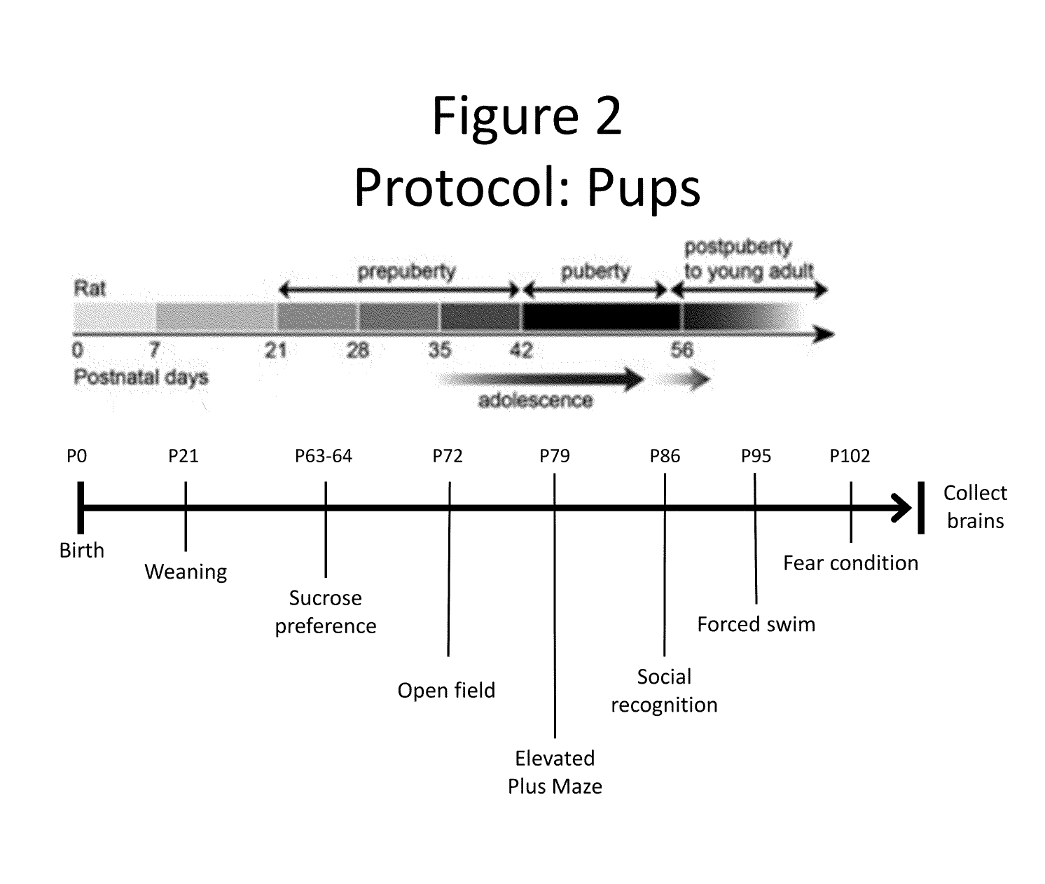

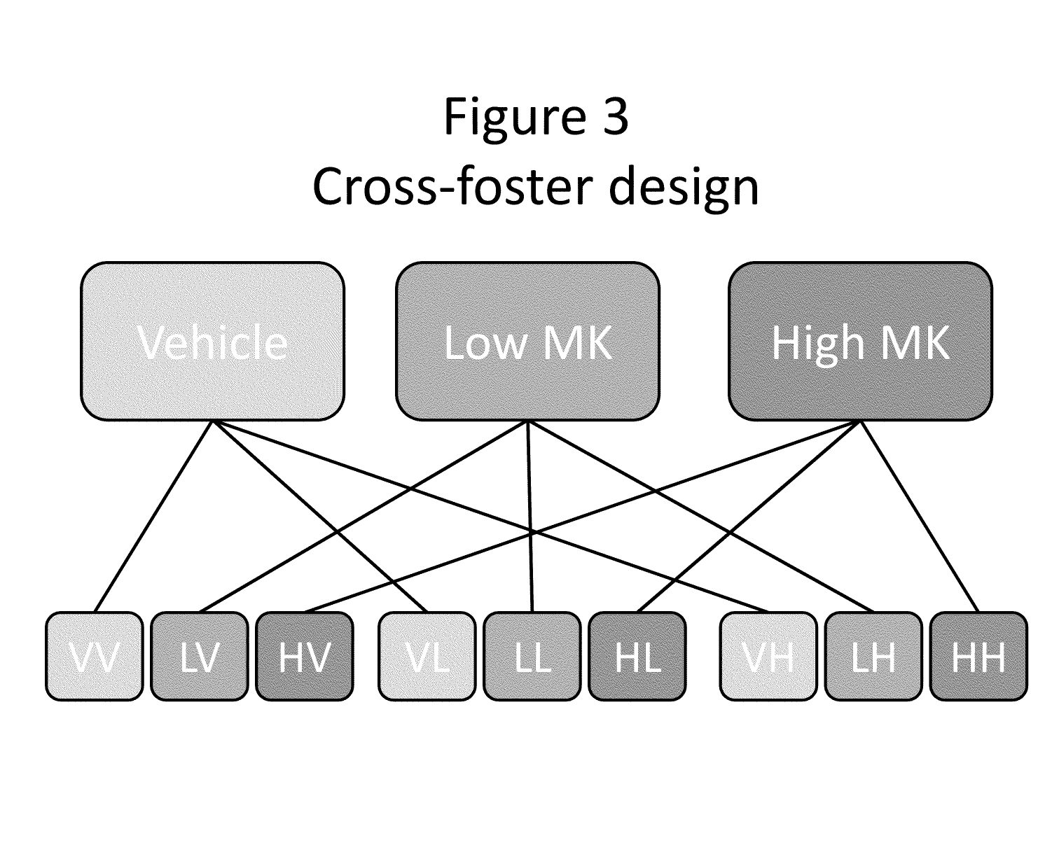

Initially, it was demonstrated that MK-0677, an orally active growth hormone (GH) secretagogue, enhanced maternal depressive behaviors. The data is shown in Figure, a bar graph depicting depressive behaviors in post-partum mothers in response to vehicle control of MK-0677 treatment. Experiments were designed to determine the effects of elevated ghrelin activity on the fetus. FIGS. 2-3 demonstrate the design of these protocols.

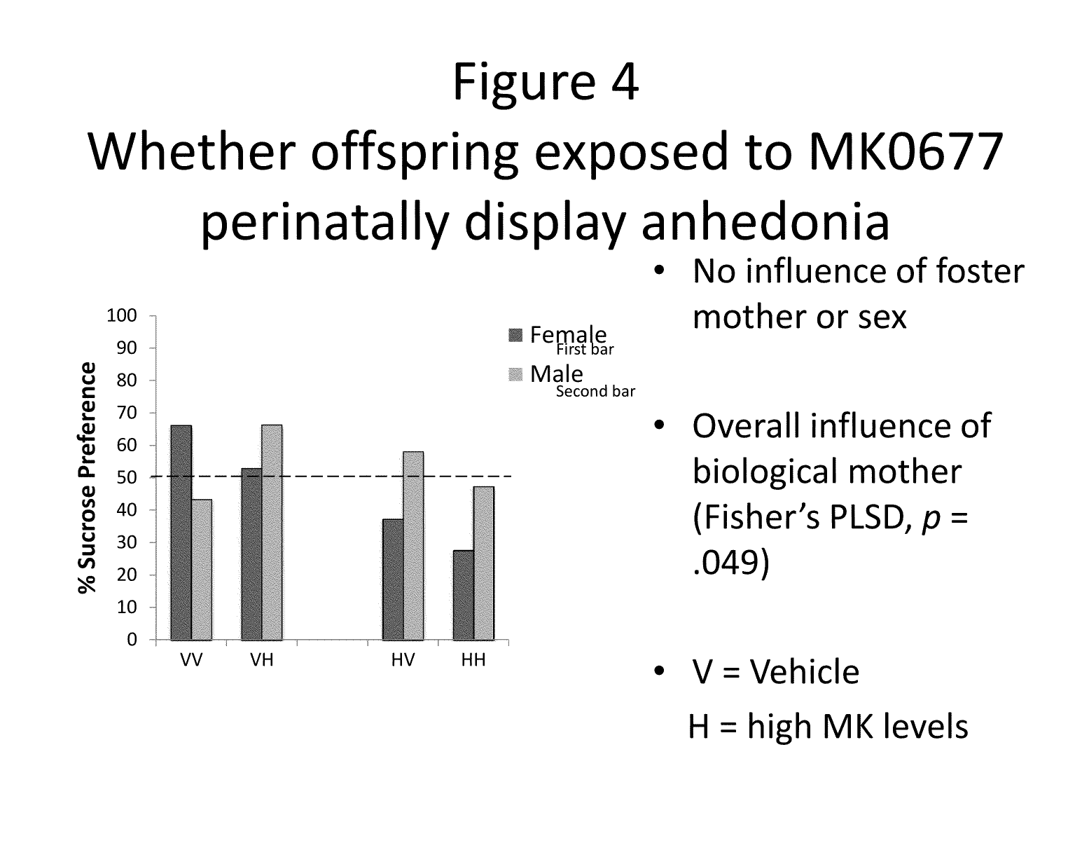

The first set of experiments was designed to determine the degree of anhedonia in offspring when raised by an MK-treated mom. Anhedonia is the inability of a subject to experience pleasure from activities usually found enjoyable. A first experiment was conducted to show the effects of perinatal exposure to MK0677 on female versus male offspring. The data is shown in FIG. 4. V refers to a vehicle control. H refers to high MK0677 exposure levels. The data demonstrate that no difference was seen between the response of male and female babies. Additionally the foster mother had no effect. Overall an influence was observed for the biological mother.

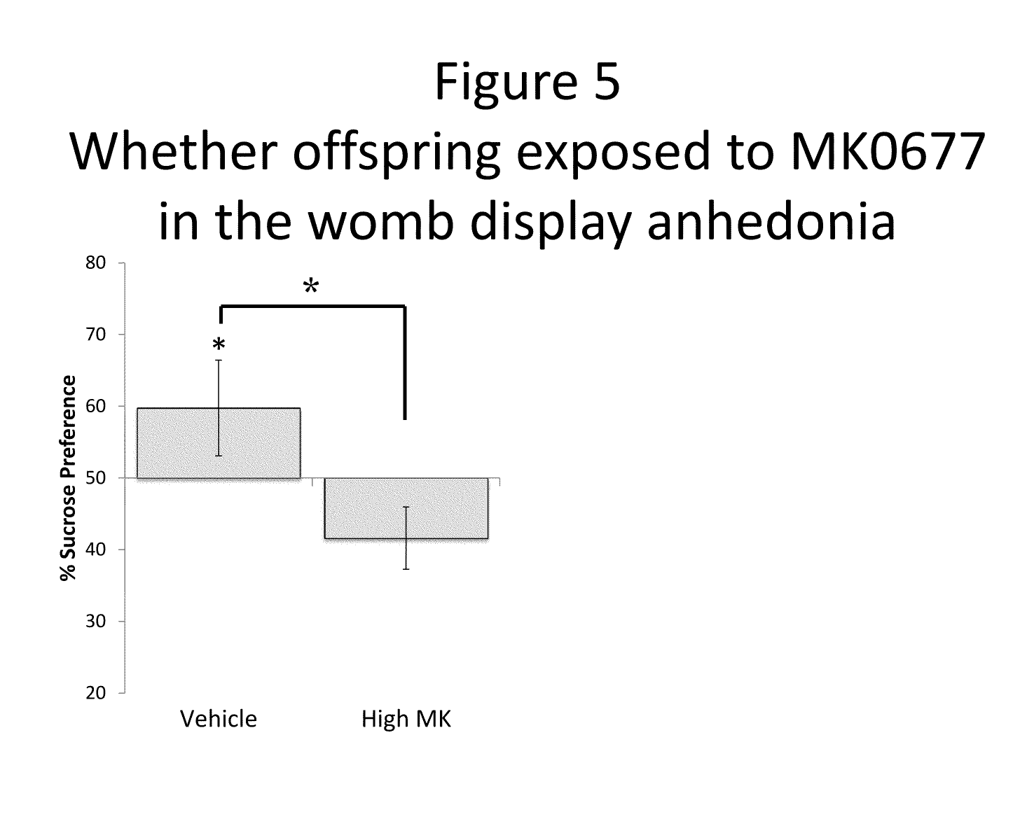

Another study involved determining the effects of exposure to MK0677 in the womb on offspring. The data is shown in FIG. 5. The results demonstrate that sucrose preference in offspring from mothers exposed to high MK levels (n=11) was significantly lower compared to controls (n=12, t-test, p=0.03). Additionally, pups from vehicle-treated mothers significantly preferred sucrose solution to water (p=0.046 vs. 50%). Pups from MK mothers did not (NS vs. 50%).

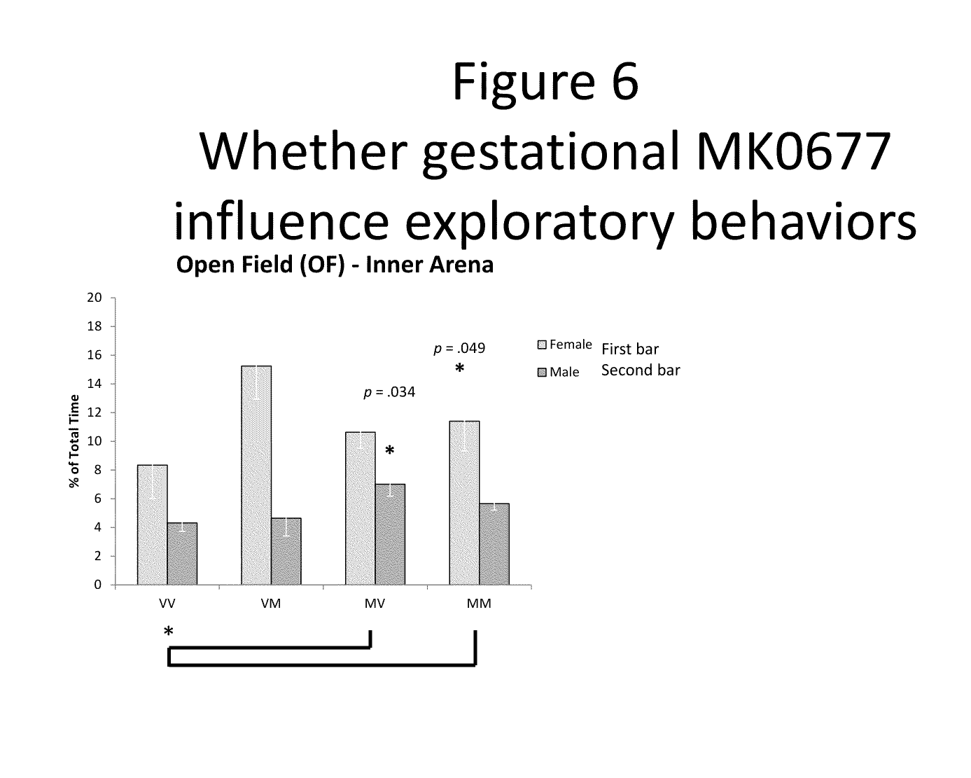

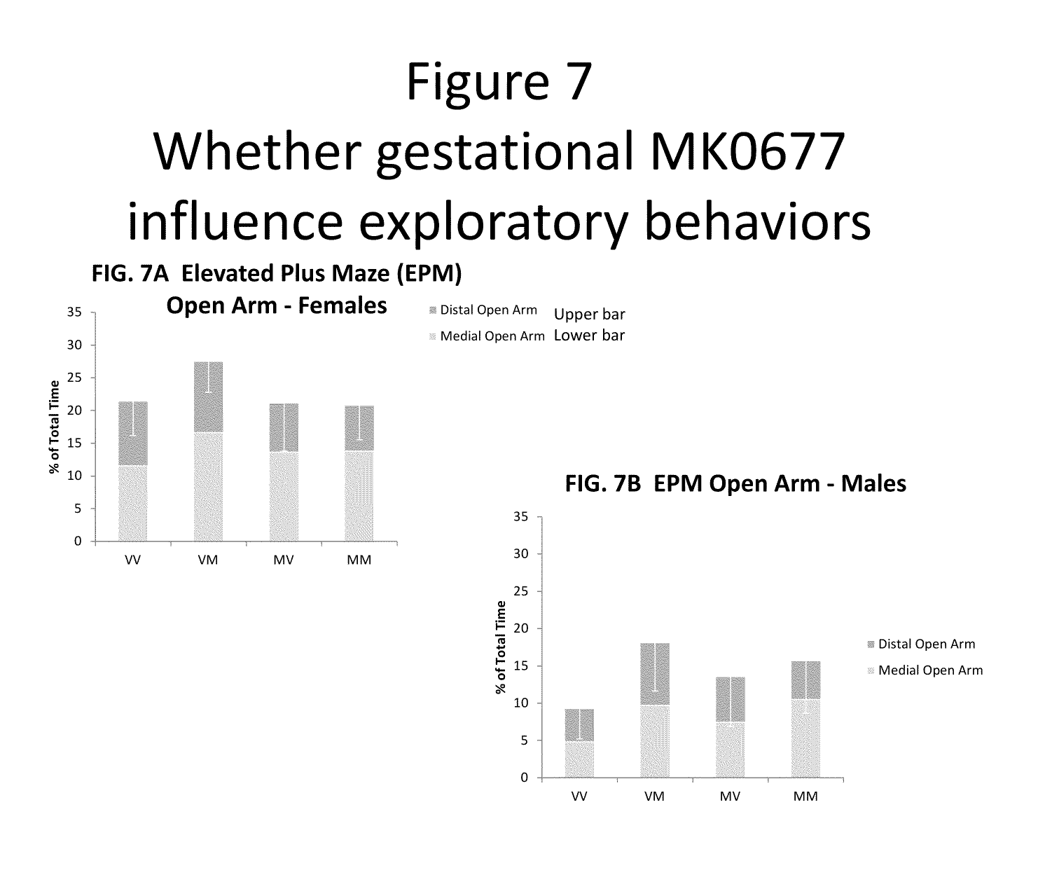

A second set of experiments was conducted to analyze risk-seeking behavior (less anxiety) in the offspring when raised by an MK-treated mom. A study examined the influence of gestational exposure to MK0677 on exploratory behaviors of female versus male offspring. The data is shown in FIG. 6, a bar graph which measures the influence of gestational exposure to MK0677 on exploratory behaviors in an Open Field inner arena. The data presented in FIGS. 7A and 7B demonstrates the influence of gestational exposure to MK0677 on exploratory behaviors of female (FIG. 7A) versus male (FIG. 7B) offspring as measured in an Elevated Plus Maze (EPM) Open Arm. The Distal Open Arm is shown in the top dark gray bar and the Medial Open Arm is shown in the lower light gray bar.

The data demonstrate changes in risk taking behavior in animals born to mothers having elevated ghrelin levels. These animals are much bigger risk takers. They are less anxious but are have risk taking behaviors and are hyper exploratory. They have a higher propensity for addiction or thrill seeking. Interestingly, the animals exposed to elevated maternal ghrelin had elevated ghrelin through their lifetime. The discovery that these animals had elevated ghrelin levels even as adults and showed altered behaviors throughout life was quite surprising. These animals show “anhedonic” behaviors (under value rewards) and aversive behavior (overestimate aversive stimuli), which are classic symptom of depression.

What is claimed is:

1. A method for treating stress-induced susceptibility to mental disorders in a fetus, comprising
determining a level of ghrelin expression in a pregnant subject and if the pregnant subject has ghrelin levels that are higher than normal ghrelin levels in a normal pregnant subject at a similar stage of pregnancy, administering to the pregnant subject an effective amount of a ghrelin antagonist to reduce functional ghrelin exposure in a fetus and to treat stress induced susceptibility to mental disorders in the fetus.

2. The method of claim 1 wherein the pregnant subject has not previously been identified as a subject that is exposed to chronic stress.

3. The method of claim 1 wherein the pregnant subject has previously been identified as a subject that is exposed to chronic stress.

4. The method of claim 1 further comprising continuing to treat the pregnant subject after the fetus is born.

5. The method of claim 1 further comprising once the fetus is born, administering to the baby an effective amount of a ghrelin antagonist, wherein the ghrelin antagonist is administered directly to the baby or through the mother's breast milk.

6. The method of claim 1 wherein the ghrelin antagonist targets the ghrelin receptor.

7. The method of claim 1 wherein the ghrelin antagonist is a GHSr1a antagonist or a GHSr1a inverse antagonist.

8. The method of claim 1 wherein the ghrelin antagonist is an anti-ghrelin vaccine.

9. The method of claim 1 wherein the ghrelin antagonist is O-acyltransferase (GOAT).

10. The method of claim 1 wherein the ghrelin antagonist is a compound that reduces or inhibits the synthesis or release of ghrelin by the stomach.

11. The method of claim 1 wherein the ghrelin antagonist is a compound that reduces or prevents ghrelin from crossing the blood-brain barrier.

12. A method for identifying a fetus at risk of developing a mental disorder, comprising
determining a level of ghrelin expression in a pregnant subject and comparing the ghrelin levels with normal ghrelin levels found in a normal pregnant subject at a similar stage of pregnancy, wherein if the pregnant subject has ghrelin levels that are higher than normal ghrelin levels, the pregnant subject has a fetus that is at risk of developing a mental disorder.

13. The method of claim 12 wherein normal ghrelin levels correspond to ghrelin levels in the pregnant subject prior to pregnancy.

14. The method of claim 12 wherein the level of ghrelin expression is assayed on a blood sample from the subject.

15. A method for treating post-partum depression in a subject, comprising
administering to a subject having or susceptible to postpartum depression an effective amount of a ghrelin antagonist to treat the postpartum depression in the subject.

16. The method of claim 15, wherein the subject has elevated ghrelin levels, relative to a normal subject.

17. The method of claim 15, wherein the subject has not previously been identified as a subject that is exposed to chronic stress.

18. The method of claim 15, wherein the subject has not previously been identified as having elevated ghrelin levels.

19. The method of claim 15, wherein the ghrelin antagonist targets the ghrelin receptor.

20. The method of claim 15, wherein the ghrelin antagonist is a GHSr1a antagonist or a GHSr1a inverse antagonist.

21. The method of claim 15, wherein the ghrelin antagonist is an anti-ghrelin vaccine.


SEQUENCE LISTING (4 sequences)

SEQ ID NO: 1 - protein, 4 aa, Artificial Sequence
  Synthetic Polypeptide
GSSF

SEQ ID NO: 2 - protein, 5 aa, Artificial Sequence
  Synthetic Polypeptide
LSPEX

SEQ ID NO: 3 - protein, 6 aa, Artificial Sequence
  Synthetic Polypeptide
AKLQPR

SEQ ID NO: 4 - protein, 8 aa, Artificial Sequence
  Synthetic Polypeptide
GSSFLSPE